(19) United States
(12) Patent Application Publication
(10) Pub. No.: US 2012/0088707 A1
(43) Pub. Date: Apr. 12, 2012

(54) ALKYL (METH) ACRYLATES COMPRISING A LONG LINEAR CHAIN BEARING ALKYL RAMIFICATIONS, PREPARATION THEREOF AND USE THEREOF IN THE PRODUCTION OF PETROLEUM ADDITIVES

(75) Inventor: Jean-Michel Paul, Metz (FR)

(73) Assignee: Arkema France, Colombes (FR)

(21) Appl. No.: 12/445,678

(22) PCT Filed: Oct. 4, 2007
(86) PCT No.: PCT/FR07/52079

§ 371 Date: Oct. 20, 2010

(30) Foreign Application Priority Data
Oct. 17, 2006 (FR) 0654320

(51) Int. Cl. C10L 1/19; C10M 129/70; C10L 1/188; C07C 69/54; C07C 67/03

(52) U.S. Cl. 508/463; 560/205; 560/217; 44/388; 44/385

(57) ABSTRACT

The invention relates to alkyl(meth)acrylates comprising a long linear chain bearing alkyl ramifications corresponding to general formula (I):

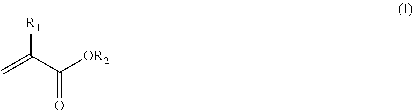

in which R₁ is a hydrogen atom or a methyl radical and R₂ is a long alkyl chain of formula (II): R(CH₂—CH₂)ₙ(CH₂—CH(R₃))ₘ— in which R is a methyl or ethyl radical, R₃ is an linear- or branched-chain alkyl radical containing from 1 to 6 carbon atoms, n=9 to 20, m=1 to 5, wherein it is possible for the —CH₂—CH₂— and —CH₂—CH(R₃)— units to be distributed in block or random form. These compounds can in particular be used for the production of additives intended to lower the settings point of paraffinic oils or fuels (diesel oils or fuel oils), and for the production of additives for lubricants.

The present invention relates to (meth)acrylic monomers and has more particularly as subject-matter alkyl(meth)acrylates comprising a long linear chain carrying alkyl branchings, and also their preparation and their use in the manufacture of petroleum additives, of fuel additives or of lubricating additives.

Alkyl(meth)acrylates comprising a long linear chain, that is to say for which the number of carbon atoms is at least 18, are used as comonomers in formulations intended for the lowering of the congealing point of paraffinic oils. This is because crude oils can comprise paraffin waxes, the nature and the content of which are directly related to the extraction site. At a temperature which depends on the nature of oils, crystallization of these paraffin waxes occurs and results in reduction in their fluidity, which is consequently harmful to their transportation and their storage. An advantageous solution consists in lowering the congealing point of these oils by addition of an additive. Thus, for example:

In French patent FR 1 575 984, the proposal was made to use macromolecular compounds of comb type constructed on the model of a hydrocarbon chain carrying fairly long side chains (of at least 14 carbon atoms). Other patents, such as French patent FR 2 128 589 and U.S. Pat. No. 2,839,512, claim the use of copolymers of C₁₈-C₃₀ acrylates, preferably C₁₈-C₂₂ acrylates, and of a heterocyclic monomer of vinylpyridine type. For its part, international application WO 97/34940 claims the use of acrylic copolymers prepared from linear C₂₄ to C₆₀ (preferably C₃₀ to C₄₀) acrylates and from C₁₀ to C₂₂ acrylates (for example behenyl acrylate).

The alkyl(meth)acrylates comprising a long linear chain of use in the context which has just been indicated are characterized by a high melting point, which results in numerous disadvantages, in particular:
The handling thereof requires that they be in and be maintained in the liquid state and thus above their melting point.
The storage thereof has to be carried out in reheated containers or in reheatable barrels or kegs, with all the risks of polymerization associated with such a storage method.
The preparation thereof employs alcohols comprising a long linear alkyl chain which are obtained by polymerization in ethylene and which are themselves characterized by a high melting point, which is problematic when they have to be introduced in the liquid form into the reactor in which the synthesis is carried out.

In order to facilitate the use of alkyl acrylates comprising a long linear chain, the proposal was made, in patent application FR 2 772 375, to mix the alkyl acrylate comprising a long linear chain with a solvent, the nature and the amount of which are chosen so that the mixture has a melting point markedly below that of the acrylate. Under this presentation, said acrylate can be packaged in the liquid form at low temperature in reheated containers without risk of polymerization or stored in barrels and melted during the subsequent use thereof in complete safety. This solution exhibits, however, the disadvantage of using a solvent, which requires specific storage and results in some restrictions.

The Applicant Company has now found that the presence of alkyl branchings in the alkyl chain of (meth)acrylates comprising a long linear chain results in products with a lower melting point than their branching-free homologs which do not exhibit the abovementioned disadvantages. The handling of these products is facilitated thereby and the risks of polymerization during the melting thereof are reduced. Furthermore, by lowering the melting point, the risks of crystallization of the product during the emptying of the reactor into pipes for transferring toward the containers or barrels are proportionately avoided. The addition of solvent in order to lower the melting point of the product is no longer necessary, which is also an advantage with regard to industrial hygiene.

A subject-matter of the present invention is thus alkyl(meth)acrylates comprising a long linear chain carrying alkyl branchings corresponding to the general formula (I):

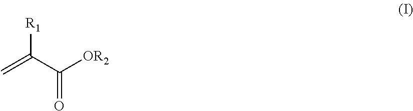

in which R₁ is a hydrogen atom or a methyl radical and R₂ represents a long alkyl chain of the formula (II):

R(CH₂—CH₂)ₙ(CH₂—CH(R₃))ₘ— (II)

in which R is a methyl or ethyl radical, R₃ is an alkyl radical comprising a linear or branched chain having from 1 to 6 carbon atoms, n=9 to 20 and m=1 to 5, it being possible for the —CH₂—CH₂— and —CH₂—CH(R₃)— units to be distributed in the block or random form.

Preferably, n=9 to 15 and m=1 to 3.

The alkyl(meth)acrylates comprising a long linear chain carrying alkyl branchings according to the invention can be provided in the form of a mixture of alkyl(meth)acrylates comprising a long linear chain carrying alkyl branchings, the alkyl chain length in the formula (I) then representing a mean length based on the distribution of the various chains.

The R₃ radical can in particular be methyl, ethyl, n-propyl, isopropyl, n-butyl, isobutyl, pentyl or hexyl; preferably, R₃ is methyl or ethyl.

Preferably, R₁ is a hydrogen atom.

More particularly, the length of the linear chain represented by the sum m+n is between 10 and 25, preferably between 10 and 17.

The alkyl(meth)acrylates of the invention generally exhibit melting points of less than 80° C., more particularly of less than 70° C., preferably of between 40° C. and 60° C. The preferred alkyl(meth)acrylates of the invention are those for which the melting point ranges from 40° C. to 50° C. They generally exist in the form of waxy solids at ambient temperature. They can be stored at ambient temperature and easily remelted at a relatively low temperature without risk of polymerization for the purpose of the use thereof.

The invention also relates to a process for the preparation of alkyl(meth)acrylates comprising a long linear chain carrying alkyl branchings corresponding to the general formula (I):

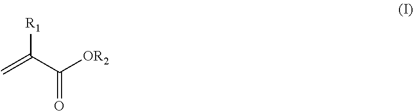

in which R₁ is a hydrogen atom or a methyl radical and R₂ represents a long alkyl chain of the formula (II):

R(CH₂—CH₂)ₙ(CH₂—CH(R₃))ₘ— (II)

in which R is a methyl or ethyl radical, R₃ is an alkyl radical comprising a linear or branched chain having from 1 to 6 carbon atoms, n=9 to 20 and m=1 to 5, it being possible for the —CH₂—CH₂— and —CH₂—CH(R₃)— units to be distributed in the block or random form, by transesterification between a light ester of formula (III):

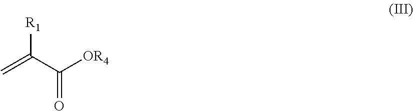

in which R₁ has the abovementioned definition and R₄ is an alkyl radical having from 1 to 4 carbon atoms, and an alcohol of the formula R₂—OH in which R₂ corresponds to the formula (II) indicated above, in the presence of at least one transesterification catalyst and of at least one polymerization inhibitor.

Mention may be made, as examples of light esters which can be used in the process according to the invention, of ethyl acrylate, butyl acrylate or methyl methacrylate.

The alcohols which can be used in the process according to the invention are alcohols comprising a long linear alkyl chain carrying alkyl branchings which are prepared, for example, from an ethylene/propylene or ethylene/butylene copolymer. The presence of branchings makes it possible to lower the melting point of the alcohols and of the (meth)acrylates which derive therefrom. Said alcohols have a lower melting point than their homologs not exhibiting branchings obtainable in particular by polymerization of ethylene. They are thus easier to handle. Their use in the preparation of the (meth)acrylates of the invention thus exhibits numerous advantages.

Mention may in particular be made, as examples of alcohols which can be used in the process according to the invention, of the products sold by Baker Petrolite under the trademark Unilin™, in particular Unilin X1187™, which exhibits a melting point of approximately 58° C. By way of comparison, Unilin 425™, with a similar molar mass but with a perfectly linear structure, exhibits a melting point of 91° C.; it results in an alkyl acrylate comprising a long linear chain with a melting point of 83° C.

The transesterification is carried out under conventional conditions, that is to say with a molar excess of light ester (III) with respect to the alcohol R₂—OH, the ester (III)/alcohol R₂—OH molar ratio generally being between 1.1 and 6 and more particularly between 1.1 and 3.5. The transesterification temperature is generally between 80° C. and 160° C. and more particularly between 80° C. and 120° C.

The reaction is carried out while bubbling through air or depleted air (for example, air comprising 8 vol % of O₂ and 92 vol % of N₂), the light alcohol R₄—OH generated during reaction being removed as it is formed in order to shift the equilibrium and light ester (III) in excess being removed by distillation after the reaction.

The transesterification is carried out in the presence of a catalyst. Mention may be made, as examples of catalysts which can be used, of: dibutyltin oxide and the other tin derivatives, including, for example, distannoxanes, titanium alkoxides, such as, for example, isopropyl titanate, alkali metal or alkaline earth metal alkoxides, such as, for example, sodium, calcium and magnesium alkoxides, hydroxides or carbonates, for example lithium, calcium, magnesium or cesium hydroxides or carbonates, calcium, zirconium or lithium acetylacetonates and other products of this family.

The amount of catalyst employed is generally between 0.001 and 0.05 mol per 100 mol of alcohol R₂—OH and preferably between 0.001 and 0.01 mol per 100 mol of alcohol R₂—OH.

The process according to the invention is carried out in the presence of at least one polymerization inhibitor chosen from products well known to a person skilled in the art. Mention may in particular be made of the following compounds: phenothiazine, hydroquinone, hydroquinone methyl ether, hindered phenols of BHT type and their homologs, or compounds carrying nitroxy groups of 4-hydroxy-TEMPO or 4-oxo-TEMPO type. The polymerization inhibitor is used in a proportion of 0.01 to 0.5% by weight, with respect to the alcohol R₂—OH.

Another subject-matter of the present invention is the use of alkyl(meth)acrylates comprising a long linear chain carrying alkyl branchings corresponding to the general formula (I) in the manufacture of additives intended for lowering the congealing point of paraffinic oils or of fuels (gas oils and fuel oils), and also in the manufacture of lubricant additives. For the purpose of this use, the alkyl(meth)acrylates comprising a long linear chain carrying alkyl branchings corresponding to the general formula (I) are employed in particular in the preparation of (meth)acrylic polymers according to any technique well known to a person skilled in the art, said polymers exhibiting properties as depressants of the pour point of paraffin crude oils or of fuels.

The following examples illustrate the present invention without, however, limiting the scope thereof.

### EXAMPLES

### Products Employed

EA: ethyl acrylate
BuA: butyl acrylate
MMA: methyl methacrylate
HQME: hydroquinone monomethyl ether
Unilin X1187™: alcohol of formula R₂OH with R₂ corresponding to the formula (II) with m+n=approximately 16; m=approximately 2; R═R₃═CH₃; distribution centered on C₃₀-C₃₂, with a number-average molar mass of approximately 450 g/mol.

### Example 1

The following are charged to a mechanically stirred reactor heated by circulation of thermostatically controlled oil inside a jacket and surmounted by a distillation column with a top condenser:
283 g of premelted alcohol Unilin X1187™
274 g of EA
0.14 g (i.e. 230 ppm, with respect to the weight of the reactants) of an inhibitor HQME.

Bubbling of air into the reaction mass is maintained throughout the duration of the synthesis.

The reaction medium is dried, the water being removed by distillation in the form of an EA/water azeotrope. 0.64 g of zirconium acetylacetonate is subsequently added as catalyst.

The reaction is carried cut under reduced pressure (5.98×10⁴-5.05×10⁴ Pa, i.e. 450-380 mmHg) at a temperature of between 90 and 105° C. The light alcohol (ethanol) is removed by distillation in the form of an EA/ethanol azeotrope in order to shift the equilibrium in the direction of the formation of the acrylate of Unilin X1187. When the reaction is complete, the excess EA is distilled off under vacuum (0.21×10⁴ Pa, i.e. 16 mmHg).

The final crude product (303 g), which is liquid and homogeneous under warm conditions, is emptied at approximately 70° C. Its viscosity at 40° C. is 34 cps. At ambient temperature, it exists in the form of a waxy solid, the melting point of which is approximately 40° C.

This product can be stored at ambient temperature and melted during the use thereof at approximately 40° C. without risk of polymerization.

### Example 2

The following are charged to the installation described in example 1:
283 g of premelted alcohol Unilin X1187™
230 g of BuA
100 g of cyclohexane
0.11 g of HQME.

Bubbling of air into the reaction mass is maintained throughout the duration of the synthesis.

The water present in the reactants is removed by distillation in the form of a cyclohexane/water azeotrope at a pressure of between 760 and 500 mmHg (i.e. 10.1×10⁴ and 6.6×10⁴ Pa).

After removing the excess cyclohexane, 0.64 g of zirconium acetylacetonate catalyst is charged.

The reaction, which generates a BuA/butanol azeotrope, is carried out at a temperature of between 100 and 120° C. under a pressure of 200 to 50 mmHg (i.e. 2.6×10⁴-0.7×10⁴ Pa).

The excess BuA is eliminated by vacuum distillation (maximum 10 mmHg, i.e. 0.13×10⁴ Pa).

The final crude product is emptied at approximately 50° C.

### Example 3

The following are charged to the installation described in example 1:
283 g of premelted alcohol Unilin X1187™
184.4 g of MMA
0.12 g of HQME.

Bubbling of air into the reaction mass is maintained throughout the duration of the synthesis.

The reaction medium is dried, the water being removed by distillation in the form of an MMA/water azeotrope. 0.64 g of zirconium acetylacetonate is subsequently added as catalyst.

The reaction is carried out under reduced pressure (4×10⁴ Pa, i.e. 300 mmHg) at a temperature of between 90 and 105° C. The light alcohol (methanol) is removed by distillation in the form of an MMA/methanol azeotrope in order to shift the equilibrium in the direction of the formation of the methacrylate of Unilin X1187. When the reaction is complete, the excess MMA is distilled off under vacuum (0.08×10⁴, i.e. 6 mmHg).

The final crude product is emptied under warm conditions at 80° C. Its melting point is approximately 48° C.

### Example 4

Example 1 can be repeated, the Unilin X1187™ being replaced by an alcohol of formula R₂OH with R₂ corresponding to the formula (II) in which m+n=approximately 13 and m=approximately 1 and R═R₃═C₂H₅. The reactor is then charged with:
450 g of premelted alcohol R₂OH
300 g of EA
0.15 g of HQME

A compound is then obtained which exists, at ambient temperature, in the form of a wax which melts at approximately 50° C.

### Example 5

Example 1 can be repeated, the Unilin X1187™ being replaced by an alcohol of formula R₂OH with R₂ corresponding to the formula (II) in which m+n=approximately 12 and m=approximately 3 and R═R₃═C₂H₅. The reactor is then charged with:
250 g of premelted alcohol R₂OH
170 g of EA
0.15 g of HQME

A compound is then obtained which exists, at ambient temperature, in the form of a wax which melts at a temperature <50° C.

These 5 examples illustrate the formation of alkyl(meth)acrylates comprising a long linear chain carrying alkyl branchings.

1. An alkyl(meth)acrylate comprising a long linear chain carrying alkyl branchings corresponding to the general formula (I): 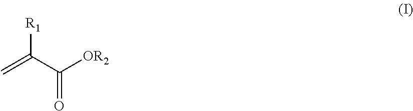
in which R₁ is a hydrogen atom or a methyl radical and R₂ represents a long alkyl chain of the formula (II):
R(CH₂—CH₂)ₙ(CH₂—CH(R₃))ₘ— (II)
in which R is a methyl or ethyl radical, R₃ is an alkyl radical comprising a linear or branched chain having from 1 to 6 carbon atoms, n=9 to 20 and m=1 to 5, wherein said —CH₂—CH₂— and —CH₂—CH(R₃)— units are distributed in block or random form.

2. The alkyl(meth)acrylate as claimed in claim 1, characterized in that, in the formula (II), n=9 to 15 and m=1 to 3.

3. The alkyl(meth)acrylate as claimed in claim 1, characterized in that, in the formula (II), m+n=10 to 17.

4. The alkyl(meth)acrylate as claimed in claim 1, characterized in that, in the formula (II), the R₃ radical is methyl or ethyl.

5. The alkyl(meth)acrylate as claimed in claim 1, characterized in that its melting point is less than 80° C.

6. The alkyl(meth)acrylate as claimed in claim 1, characterized in that its melting point is between 40° C. and 60° C.

7. A process for the preparation of alkyl(meth)acrylates comprising a long linear chain carrying alkyl branchings corresponding to the general formula (I): 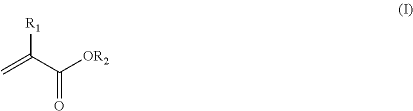
in which R₁ is a hydrogen atom or a methyl radical and R₂ represents a long alkyl chain of the formula (II):
R(CH₂—CH₂)ₙ(CH₂—CH(R₃))ₘ— (II)
in which R is a methyl or ethyl radical, R₃ is an alkyl radical comprising a linear or branched chain having from 1 to 6 carbon atoms, n=9 to 20 and m=1 to 5, it being possible for the —CH₂—CH₂— and —CH₂—CH(R₃)— units to be distributed in the block or random form,
comprising transesterifying a light ester of formula (III): 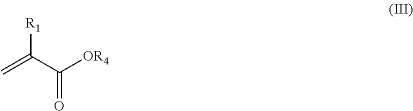
in which R₁ has the abovementioned definition and R₄ is an alkyl radical having from 1 to 4 carbon atoms,
and an alcohol of the formula R₂—OH in which R₂ corresponds to the formula (II) indicated above, in the presence of at least one transesterification catalyst and of at least one polymerization inhibitor.

8-9. (canceled)

10. A method of lowering the congealing point of paraffinic oils or fuels selected from gas oils and fuels oils, comprising adding a alkyl(meth)acrylates comprising a long linear chain carrying alkyl branchings corresponding to the general formula (I): 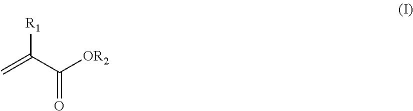
in which R₁ is a hydrogen atom or a methyl radical and R₂ represents a long alkyl chain of the formula (II):
R(CH₂—CH₂)ₙ(CH₂—CH(R₃))ₘ— (II)
in which R is a methyl or ethyl radical, R₃ is an alkyl radical comprising a linear or branched chain having from 1 to 6 carbon atoms, n=9 to 20 and m=1 to 5 wherein said —CH₂—CH₂— and —CH₂—CH(R₃)— units are distributed in block or random form.

11. Lubricants additives comprising long linear chain carrying alkyl branchings corresponding to the general formula (I): 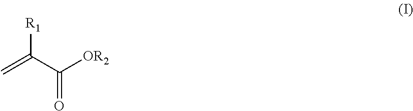
in which R₁ is a hydrogen atom or a methyl radical and R₂ represents a long alkyl chain of the formula (II):
R(CH₂—CH₂)ₙ(CH₂—CH(R₃))ₘ— (II)
which R is a methyl or ethyl radical, R₃ is an alkyl radical comprising a linear or branched chain having from 1 to 6 carbon atoms, n=9 to 20 and m=1 to 5 wherein said —CH₂—CH₂— and —CH₂—CH(R₃)— units are distributed in block or random form.
